(19) United States
(12) Patent Application Publication
(10) Pub. No.: US 2022/0233181 A1
(43) Pub. Date: Jul. 28, 2022

(54) BIOPSY TOOL HAVING PRE-APPLIED VACUUM FORCE

(71) Applicant: Michael Jay Phillips, Beaufort, NC (US)

(72) Inventor: Michael Jay Phillips, Beaufort, NC (US)

(21) Appl. No.: 17/156,950

(22) Filed: Jan. 25, 2021

(51) Int. Cl. A61B 10/02

(52) U.S. Cl. CPC A61B 10/0283

(57) ABSTRACT

A fine needle aspiration biopsy tool includes a pressurized chamber having a vacuum applied to the chamber. An actuation valve engaged by the chamber and has actuation grips on opposing sides that cooperatively allow for selective fluid engagement with an environment outside of the chamber. A port extends from the actuation valve and selectively receives a needle or a cap. In operation, the actuation valve is actuated by pressing engagement of the actuator grips to apply vacuum to the needle from the vacuum chamber to draw in fluid or mass in flow communication with an inflow end of the needle during a biopsy procedure.

### TECHNICAL FIELD

The invention herein pertains to a biopsy tool having a pre-applied vacuum force.

### BACKGROUND

When a suspicious tissue mass is discovered in a patient's breast, neck, or other area through examination, ultrasound, MRI, X-ray imaging or the like, it is often necessary to perform a biopsy procedure to remove one or more samples of that tissue in order to determine whether the mass contains cancerous cells. A biopsy may be performed using an open or percutaneous method.

An open biopsy is performed by making a large incision in the breast and removing either the entire mass, called an excisional biopsy, or a substantial portion of it, known as an incisional biopsy. An open biopsy is a surgical procedure that is usually done as an outpatient procedure in a hospital or a surgical center, involving both high cost and a high level of trauma to the patient. Open biopsy carries a relatively higher risk of infection and bleeding than does percutaneous biopsy, and the disfigurement that sometimes results from an open biopsy may make it difficult to read future mammograms. Further, the aesthetic considerations of the patient make open biopsy even less appealing due to the risk of disfigurement. Given that a high percentage of biopsies show that the suspicious tissue mass is not cancerous, the downsides of the open biopsy procedure render this method inappropriate in many cases.

Percutaneous biopsy, to the contrary, is much less invasive than open biopsy. Percutaneous biopsy may be performed using fine needle aspiration (FNA) or core needle biopsy. In FNA, a very thin needle is used to withdraw fluid and cells from the suspicious tissue mass. This method has an advantage in that it is very low-pain, so low-pain that local anesthetic is not always used because the application of it may be more painful than the FNA itself. However, a shortcoming of FNA is that only a small number of cells are obtained through the procedure, rendering it relatively less useful in analyzing the suspicious tissue and making an assessment of the progression of the cancer less simple if the sample is found to be malignant.

During a core needle biopsy, a small tissue sample is removed allowing for a pathological assessment of the tissue, including an assessment of the progression of any cancerous cells that are found. This is often accomplished with vacuum assistance.

While the vacuum assistance has a number of benefits, some practitioners prefer to perform core biopsy procedures with simpler devices that do not include a control module with graphical user interface, electronic control, vacuum generation and control, and other features. In addition to the desire to reduce capital costs, it is also generally desirable to reduce the need to tether a hand-held biopsy device to sources of mechanical motion, vacuum supply, electrical power and control. Such tethers may tend to impede positioning of the biopsy device, introduce tripping hazards, and increase set up time.

One of the other persistent problems with FNA is that conventional systems are not ergonomically appropriate and negatively impact the fine motor skills of the operator. In some systems, a pull trigger is activated by pulling onto a sliding trigger while a back portion is in the user's palm, which creates an unintended arcuate hand movement for the user as well. This is occurring while the needle is currently within the subject, which can create loss of biopsy material, maldeployment, discomfort or even injury to the subject.

### SUMMARY

This summary is provided to briefly introduce concepts that are further described in the following detailed descriptions. This summary is not intended to identify key features or essential features of the claimed subject matter, nor is it to be construed as limiting the scope of the claimed subject matter.

According to one or more embodiments, a fine needle aspiration biopsy tool includes a pressurized chamber having a vacuum applied to the chamber; an actuation valve engaged by the chamber and having actuation grips on opposing sides that cooperatively allow for selective fluid engagement with an environment outside of the chamber; and a port extending from the actuation valve that selectively receives a needle or a cap. In operation, the actuation valve is actuated by pressing engagement of the actuator grips to apply vacuum to the needle from the chamber to draw in fluid or mass in flow communication with an inflow end of the needle during a biopsy procedure.

According to one or more embodiments, the vacuum applied to the chamber is prevacuumed.

According to one or more embodiments, the vacuum applied to the chamber is provided through an outlet on the chamber that is pressure coupled with an external vacuum.

According to one or more embodiments, the actuation valve is activated by pressing on actuation grips.

According to one or more embodiments, the actuation valve is activated by twisting of the actuation valve.

According to one or more embodiments, the actuation valve is activated by actuation of one of the grips.

According to one or more embodiments, the actuation valve is configured for graduated release of vacuum, such that the user may activate desired amounts of vacuum.

According to one or more embodiments, the chamber includes graduation lines for measuring an amount of fluid vacuumed into the chamber from the biopsy procedure.

According to one or more embodiments, the tool is provided in a kit having a case, the case further carrying one or more of the needles, cleaning pads, and the cap.

According to one or more embodiments, the pressurized chamber is a syringe, and the syringe is operator controlled to apply the vacuum.

According to one or more embodiments, a method includes positioning a needle into a biopsy site, applying a vacuum to the needle to draw in fluid or mass, wherein the vacuum is applied from a pressurized chamber having a vacuum applied thereto, the vacuum having been pre-applied to the pressurized chamber, and activating an actuation valve engaged by the chamber and having actuation grips on opposing sides that cooperatively allow for selective fluid engagement with an environment outside of the chamber. In operation, the actuation valve is actuated by pressing engagement of the actuator grips to apply vacuum to the needle from the chamber to draw in fluid or mass in flow communication with an inflow end of the needle during a biopsy procedure.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated.

FIG. 1 illustrates a side view of a biopsy tool according to one or more embodiments of the presently disclosed subject matter.

FIG. 2 illustrates a top view of a biopsy tool kit according to one or more embodiments of the presently disclosed subject matter.

FIG. 3 illustrates a side view of a biopsy tool being deployed for use with a patient according to one or more embodiments of the presently disclosed subject matter.

FIG. 4 illustrates a side view of a biopsy tool according to one or more embodiments of the presently disclosed subject matter where the biopsy tool is shown having graduation lines.

FIG. 5 illustrates a side view of a biopsy tool according to one or more embodiments of the presently disclosed subject matter.

FIG. 6 illustrates a side view of a biopsy tool according to one or more embodiments of the presently disclosed subject matter.

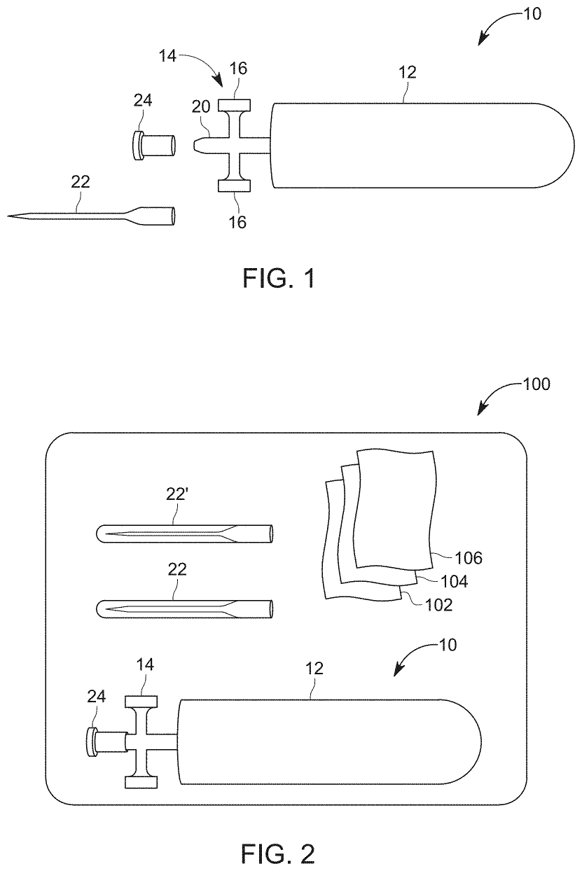

### DETAILED DESCRIPTION OF THE EMBODIMENTS

The following description and figures are illustrative and are not to be construed as limiting. Numerous specific details are described to provide a thorough understanding of the disclosure. In certain instances, however, well-known, or conventional details are not described in order to avoid obscuring the description. Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that same thing can be said in more than one way.

A fine needle aspiration biopsy tool is generally designated 10. The multiple advantages of the biopsy tool 10 are described herein, and apparent from review of the disclosure. The tool 10 includes a pressurized chamber 12 having a vacuum applied to the chamber 12. The chamber 12 is shown as a container approximating a cylindrical vial, but can take on many shapes and sizes or configurations. The pressurized chamber 12 has enough vacuum provided to create suction to perform a biopsy operation as will be described further herein.

The chamber 12 is engaged with an actuation valve 14 engaged by the chamber 12. The actuation valve 14 allows for selective fluid engagement with an environment outside of the chamber 12. In this manner, the actuation valve 14 allows the vacuum in the pressurized chamber 12 to act on an outside environment. In one or more embodiments, the actuation valve 14 includes having actuation grips 16 on opposing sides. The actuation grips 16 allow for selective actuation by an operator, such as a nurse or physician, that allows for the vacuum to act on the outside environment. The actuation valve 14 is activated by pressing on actuation grips 16 in some embodiments, circular twisting in other embodiments, and any other desired configuration. In one or more embodiments, the actuation valve 14 is configured for graduated release of vacuum, such that the user may activate desired amounts of vacuum.

A port 20 extends from the actuation valve 14. The port is in fluid connection with the chamber 12. The port 20 may selectively receive a needle 22 or a cap 24 as will be described further herein. In operation, the actuation valve 14 is actuated by pressing engagement of the actuation grips 16 to apply vacuum to the needle 22 from the vacuum chamber 12 to draw in fluid or mass in flow communication with an inflow end of the needle 22 during a biopsy procedure. The cap 24 can be removed and reapplied to prevent contamination of the tool 10.

In one or more embodiments, the vacuum applied to the chamber 12 is prevacuumed, meaning it has been applied to the chamber 12 during manufacture. Alternatively, the vacuum may be applied by a syringe as is illustrated in FIG. 6. In this manner, the chamber 12 may have further sealing characteristics such as a puncture seal or similar that can maintain vacuum until the actuation valve 14 is engaged with the chamber. Alternatively, the chamber 12 and actuation valve 14 may be provided during manufacture as a singular unit, or the actuation valve 14 may be selectively engaged with the chamber 12.

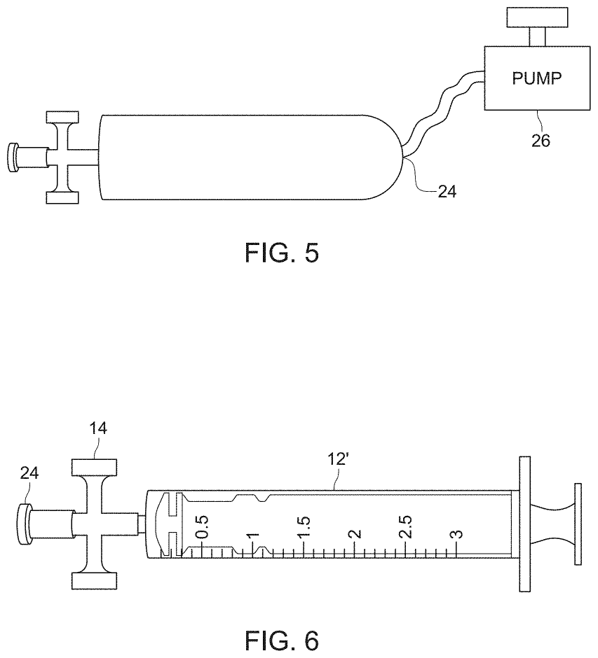

In one or more embodiments, as illustrated in FIG. 5, the vacuum applied to the chamber 12 is provided through an outlet 24 on the vacuum chamber 12 that is pressure coupled with an external vacuum pump 26. This further allows for selective removal of material in the chamber 12 after a biopsy procedure.

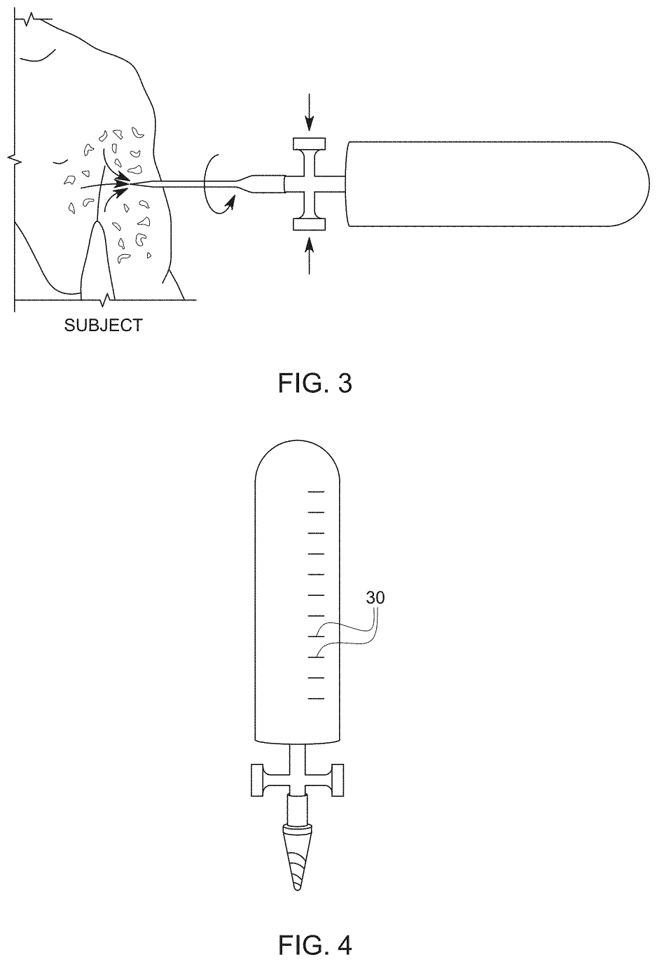

As illustrated in FIG. 4, in some embodiments, graduation lines for measuring an amount of fluid vacuumed into the chamber 12 from the biopsy procedure. Usually though, the biopsy material will be held within needle 22 for removal from the tool 10.

As illustrated in FIG. 2, the tool 10 may be provided in a kit 100 having a case, the case further carrying one or more of the needles 22, 22′, cleaning pads 102, gauze 104, and the cap 24. In this manner, the tool 10 and kit 100 are provided in a convenient to access and carry, one time use manner. The case may further include identifying info such as a QR code or other identifier associated with the patient or procedure. Alternatively, the chamber 12 may include the identifier.

FIG. 6 illustrates an alternate embodiment of the disclosure provided herein. The alternate embodiment illustrates a syringe 12′ that provides the vacuum force. As such, the syringe 12′ can replace the chamber described in this disclosure. The syringe 12′ may be conventional with a threaded outflow port, with the threaded outflow port being threadably or otherwise engaged with actuation valve 14. The remaining components of the assembly are consistent with what has been described herein.

As described, one of the persistent problems with FNA is that conventional systems are not ergonomically appropriate and negatively impact the fine motor skills of the operator. In some systems, a pull trigger is activated by pulling onto a sliding trigger while a back portion is in the user's palm, which creates an unintended arcuate hand movement for the user as well. This is occurring while the needle is currently within the subject, which can create loss of biopsy material, maldeployment, discomfort or even injury to the subject. In this manner, the disclosed invention, particularly that described in FIG. 6 allows the operator to apply the vacuum to the syringe 12′ by pulling back on the plunger. From then, the operator can apply vacuuming forces to the syringe 12′, but then operate the device by simply activating the actuation valve 14. In this manner, the application of vacuum force to the reservoir by operator pulling on a plunger associated with FNA occurs before the actuation valve 14 is then activated. In this manner, the significant forces required with conventional FNA are avoided and a finer application of vacuum can be provided for a biopsy procedure.

A method of conducting a biopsy is thus provided. The method includes positioning a needle into a biopsy site. One example of this is illustrated in FIG. 3. The method further includes applying a vacuum to the needle to draw in fluid or mass. The vacuum is applied from a pressurized chamber 12 having a vacuum applied thereto as described herein. The vacuum has been pre-applied to the pressurized chamber 12, and may be a syringe 12′ as shown in FIG. 6. The method further includes activating an actuation valve 14 engaged by the chamber and having actuation grips 16 on opposing sides that cooperatively allow for selective fluid engagement with an environment outside of the chamber 12. In operation, the actuation valve 14 is actuated by pressing engagement of the actuator grips 16 to apply vacuum to the needle 22 from the vacuum chamber 12 to draw in fluid or mass in flow communication with an inflow end of the needle 22 during a biopsy procedure.

These and other changes can be made to the disclosure in light of the Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

What is claimed is:

1. A fine needle aspiration biopsy tool, comprising:
a pressurized chamber having a vacuum applied to the chamber;
an actuation valve engaged by the chamber and having actuation grips on opposing sides that cooperatively allow for selective fluid engagement with an environment outside of the chamber;
a port extending from the actuation valve that selectively receives a needle or a cap;
wherein, in operation, the actuation valve is actuated by pressing engagement of the actuator grips to apply vacuum to the needle from the chamber to draw in fluid or mass in flow communication with an inflow end of the needle during a biopsy procedure.

2. The tool according to claim 1, wherein the vacuum applied to the chamber is prevacuumed.

3. The tool according to claim 1, wherein the vacuum applied to the chamber is provided through an outlet on the chamber that is pressure coupled with an external vacuum.

4. The tool according to claim 1, wherein the actuation valve is activated by pressing on actuation grips.

5. The tool according to claim 1, wherein the actuation valve is activated by twisting of the actuation valve.

6. The tool according to claim 1, wherein the actuation valve is activated by actuation of one of the grips.

7. The tool according to claim 1, wherein the actuation valve is configured for graduated release of vacuum, such that the user may activate desired amounts of vacuum.

8. The tool according to claim 1, wherein the chamber includes graduation lines for measuring an amount of fluid vacuumed into the chamber from the biopsy procedure.

9. The tool according to claim 1, wherein the tool is provided in a kit having a case, the case further carrying one or more of the needles, cleaning pads, and the cap.

10. The tool according to claim 1, wherein the pressurized chamber is a syringe, and the syringe is operator controlled to apply the vacuum.

11. A method of conducting a biopsy, the method comprising:
positioning a needle into a biopsy site;
applying a vacuum to the needle to draw in fluid or mass, wherein the vacuum is applied from a pressurized chamber having a vacuum applied thereto, the vacuum having been pre-applied to the pressurized chamber;
activating an actuation valve engaged by the chamber and having actuation grips on opposing sides that cooperatively allow for selective fluid engagement with an environment outside of the chamber,
wherein, in operation, the actuation valve is actuated by pressing engagement of the actuator grips to apply vacuum to the needle from the chamber to draw in fluid or mass in flow communication with an inflow end of the needle during a biopsy procedure.
